(19) United States
(12) Patent Application Publication
(10) Pub. No.: US 2011/0130662 A1
(43) Pub. Date: Jun. 2, 2011

(54) ULTRASOUND 3D SCANNING GUIDANCE AND RECONSTRUCTION METHOD AND DEVICE, AND ULTRASOUND SYSTEM

(76) Inventors: Liang Shen, Wuxi (CN); Huiren Chen, Wuxi (CN)

(21) Appl. No.: 12/956,841

(22) Filed: Nov. 30, 2010

(30) Foreign Application Priority Data
Nov. 30, 2009 (CN) 200910225864.2

(51) Int. Cl. A61B 8/14

(52) U.S. Cl. 600/443

(57) ABSTRACT

An ultrasound 3D scanning guidance and reconstruction method includes a scanning step for performing a multi-point scanning on an organ to obtain a plurality of 3D images, each 3D image containing corresponding feature information, and a reconstructing step for reconstructing a 3D image of the whole organ from these 3D images.

### CROSS REFERENCE TO RELATED APPLICATIONS

This application claims the benefit of Chinese Patent Application No. 200910225864.2 filed Nov. 30, 2009, which is hereby incorporated by reference in its entirety.

### BACKGROUND OF THE INVENTION

The present invention generally relates to the technical field of ultrasound 3D scanning, and in particular to an ultrasound 3D scanning guidance and reconstruction method and device and its system.

In the field of current ultrasound 3D scanning technology, the state-of-the-art techniques mainly focus on system implementation, such as China patent application No. 200510006818.5 and China patent application No. 02829603.

However, in practical applications, due to such factors as probe size, power limitation and etc, a frame of 3D ultrasound image is often limited in terms of scanning scope and many times cannot completely scan the whole organ being scanned. In such cases, clinic doctors often need to move an ultrasound probe to scan different positions. This can miss detecting some slices, thereby influencing the clinical diagnosis and sometimes adversely affecting the treatment.

### BRIEF DESCRIPTION OF THE INVENTION

Embodiments of the present invention provide an ultrasound 3D scanning guidance and reconstruction method and device and its system, which is capable of scanning at multiple points and then reconstructing an ultrasound 3D image of the whole organ from the 3D scanning images of different positions.

In order to solve the above problem, the technical solution of the ultrasound 3D scanning guidance and reconstruction method of the present invention is as follows: a scanning step: performing a multi-point scanning on an organ to obtain a plurality of 3D images, each 3D image containing corresponding feature information; a reconstructing step: reconstructing a 3D image of the whole organ from these 3D images.

Said feature information comprises feature blood vessel and tissue modality.

Wherein, said reconstructing step further comprises: an extracting step, for extracting said feature information of two adjacent 3D images; a coordinate system transformation step, for using said feature information, transforming the coordinates of a point in one 3D image into coordinates under the coordinate system of the other 3D image; and repeating the above steps until all 3D images are under a single coordinate system.

Said coordinate system transformation step further comprises: finding four common feature points in these two 3D images, said feature points being points in space containing feature information; for each feature point W, listing the following three equations respectively:

x′_{w}=R₁₁*x_{w}+R₁₂*y_{w}+R₁₃*z_{w}+tₓ

y′_{w}=R₂₁*x_{w}+R₂₂*y_{w}+R₂₃*z_{w}+t_{y}

z′_{w}=R₃₁*x_{w}+R₃₂*y_{w}+R₃₃*z_{w}+t_{z}

wherein (x_{w},y_{w},z_{w}) is the coordinates of feature point w under the coordinate system of one 3D image, and (x′_{w},y′_{w},z′_{w}) is the coordinates of feature point w under the coordinate system of the other 3D image;

The coordinate system transformation system also includes obtaining the values of 12 unknown parameters R₁₁, R₁₂, R₁₃, R₂₁, R₂₂, R₂₃, R₃₁, R₃₂, R₃₃, tₓ, t_{y} and t_{z} from the listed 12 equations; substituting the resulting 12 unknown parameters into the following equation and performing coordinate system transformation according to the following equation:

$$[ \begin{matrix}{x}^{′} \\ {y}^{′} \\ {z}^{′} \\ 1\end{matrix} ] = [ \begin{matrix}{R}_{11} & {R}_{12} & {R}_{13} & {t}_{x} \\ {R}_{21} & {R}_{22} & {R}_{23} & {t}_{y} \\ {R}_{31} & {R}_{32} & {R}_{33} & {t}_{z} \\ 0 & 0 & 0 & 1\end{matrix} ] [ \begin{matrix}x \\ y \\ z \\ 1\end{matrix} ]$$

wherein (x,y,z) is the coordinates of a point on one 3D image under the coordinate system of this 3D image; and (x′,y′,z′) is the coordinates of said point under the coordinate system of the other 3D image.

The ultrasound 3D scanning guidance and reconstruction method of the present invention further comprises a verifying step for verifying whether the mosaicing error of the 3D image of the whole organ resulting from mosaicing is within an acceptable range.

Said verifying step further comprises: selecting L common feature points in a 3D image m and a 3D image n; calculating said mosaicing error according to the following equation:

$$ɛ = {\sum }_{i = 1}^{L} \sqrt{{( {x}_{mi} - {x}_{ni} )}^{2} + {( {y}_{mi} - {y}_{ni} )}^{2} + {( {z}_{mi} - {z}_{ni} )}^{2}}$$

wherein (xₘᵢ,yₘᵢ,zₘᵢ) is the coordinates of feature point i under the coordinate system of the 3D image m; and (xₙᵢ,yₙᵢ,zₙᵢ) is the coordinates of said feature point i under the coordinate system of the 3D image n; and judging whether said mosaicing error ε is less than a threshold.

Correspondingly, the technical solution of the ultrasound 3D scanning guidance and reconstruction device of the present invention comprises: a scanning unit, for performing a multi-point scanning on an organ to obtain a plurality of 3D images, each 3D image containing corresponding feature information; a reconstructing unit, for reconstructing a 3D image of the whole organ from these 3D images.

Said feature information comprises feature blood vessel and tissue modality.

Said reconstructing unit further comprises: an extracting unit, for extracting said feature information of two adjacent 3D images; a coordinate system transformation unit, for using said feature information, transforming the coordinates of a point in one 3D image into coordinates under the coordinate system of the other 3D image.

Said coordinate system transformation unit further comprises: a unit for finding four common feature points in these two 3D images, said feature points being points in space containing feature information; a unit for listing the following three equations respectively for each feature point W:

x′_{w}=R₁₁*x_{w}+R₁₂*y_{w}+R₁₃*z_{w}+tₓ

y′_{w}=R₂₁*x_{w}+R₂₂*y_{w}+R₂₃*z_{w}+t_{y}

z′_{w}=R₃₁*x_{w}+R₃₂*y_{w}+R₃₃*z_{w}+t_{z}

wherein (x_{w},y_{w},z_{w}) is the coordinates of the feature point w under the coordinate system of one 3D image, and (x′_{w},y′_{w},z′_{w}) is the coordinates of the feature point w under the coordinate system of the other 3D image;

The coordinate system transformation unit also includes a unit for obtaining the values of 12 unknown parameters R₁₁, R₁₂, R₁₃, R₂₁, R₂₂, R₂₃, R₃₁, R₃₂, R₃₃, tₓ, t_{y} and t_{z} from the listed 12 equations; and a unit for substituting the resulting 12 unknown parameters into the following equation and performing coordinate system transformation according to the following equation:

$$[ \begin{matrix}{x}^{′} \\ {y}^{′} \\ {z}^{′} \\ 1\end{matrix} ] = [ \begin{matrix}{R}_{11} & {R}_{12} & {R}_{13} & {t}_{x} \\ {R}_{21} & {R}_{22} & {R}_{23} & {t}_{y} \\ {R}_{31} & {R}_{32} & {R}_{33} & {t}_{z} \\ 0 & 0 & 0 & 1\end{matrix} ] [ \begin{matrix}x \\ y \\ z \\ 1\end{matrix} ]$$

wherein (x,y,z) is the coordinates of a point on one 3D image under the coordinate system of this 3D image; and (x′,y′,z′) is the coordinates of said point under the coordinate system of the other 3D image.

Moreover, the ultrasound 3D scanning guidance and reconstruction device of the present invention further comprises a verifying unit for verifying whether the mosaicing error of the 3D image of the whole organ resulting from mosaicing is within an acceptable range.

Said verifying unit further comprises: a unit for selecting L common feature points in a 3D image m and a 3D image n; a unit for calculating said mosaicing error according to the following equation:

$$ɛ = {\sum }_{i = 1}^{L} \sqrt{{( {x}_{mi} - {x}_{ni} )}^{2} + {( {y}_{mi} - {y}_{ni} )}^{2} + {( {z}_{mi} - {z}_{ni} )}^{2}}$$

wherein (xₘᵢ,yₘᵢ,zₘᵢ) is the coordinates of feature point i under the coordinate system of the 3D image m; and (xₙᵢ,yₙᵢ,zₙᵢ) is the coordinates of said feature point i under the coordinate system of the 3D image n; and a unit for judging whether said mosaicing error ε is less than a threshold.

Further, the ultrasound system of the present invention comprises an ultrasound 3D scanning guidance and reconstruction device, said ultrasound 3D scanning guidance and reconstruction device comprising: a scanning unit, for performing a multi-point scanning on an organ to obtain a plurality of 3D images, each 3D image containing corresponding feature information; a reconstructing unit, for reconstructing a 3D image of the whole organ from these 3D images.

Said feature information comprises feature blood vessel and tissue modality.

Said reconstructing unit further comprises: an extracting unit, for extracting said feature information of two adjacent 3D images; a coordinate system transformation unit, for using said feature information, transforming the coordinates of a point in one 3D image into coordinates under the coordinate system of the other 3D image.

Said coordinate system transformation unit further comprises: a unit for finding four common feature points in these two 3D images, said feature points being points in space containing feature information; a unit for listing the following three equations respectively for each feature point W:

x′_{w}=R₁₁*x_{w}+R₁₂*y_{w}+R₁₃*z_{w}+tₓ

y′_{w}=R₂₁*x_{w}+R₂₂*y_{w}+R₂₃*z_{w}+t_{y}

z′_{w}=R₃₁*x_{w}+R₃₂*y_{w}+R₃₃*z_{w}+t_{z}

wherein (x_{w},y_{w},z_{w}) is the coordinates of feature point w under the coordinate system of one 3D image, and (x′_{w},y′_{w},z′_{w}) is the coordinates of the feature point w under the coordinate system of the other 3D image;

The coordinate system transformation unit also includes a unit for obtaining the values of 12 unknown parameters R₁₁, R₁₂, R₁₃, R₂₁, R₂₂, R₂₃, R₃₁, R₃₂, R₃₃, tₓ, t_{y} and t_{z} from the listed 12 equations; and a unit for substituting the resulting 12 unknown parameters into the following equation and performing coordinate system transformation according to the following equation:

$$[ \begin{matrix}{x}^{′} \\ {y}^{′} \\ {z}^{′} \\ 1\end{matrix} ] = [ \begin{matrix}{R}_{11} & {R}_{12} & {R}_{13} & {t}_{x} \\ {R}_{21} & {R}_{22} & {R}_{23} & {t}_{y} \\ {R}_{31} & {R}_{32} & {R}_{33} & {t}_{z} \\ 0 & 0 & 0 & 1\end{matrix} ] [ \begin{matrix}x \\ y \\ z \\ 1\end{matrix} ]$$

wherein (x,y,z) is the coordinates of a point on one 3D image under the coordinate system of this 3D image; and (x′,y′,z′) is the coordinates of said point under the coordinate system of the other 3D image.

Moreover, the ultrasound system of the present invention further comprises a verifying unit for verifying whether the mosaicing error of the 3D image of the whole organ resulting from mosaicing is within an acceptable range.

Said verifying unit further comprises: a unit for selecting L common feature points in a 3D image m and a 3D image n; a unit for calculating said mosaicing error according to the following equation:

$$ɛ = {\sum }_{i = 1}^{L} \sqrt{{( {x}_{mi} - {x}_{ni} )}^{2} + {( {y}_{mi} - {y}_{ni} )}^{2} + {( {z}_{mi} - {z}_{ni} )}^{2}}$$

wherein (xₘᵢ,yₘᵢ,zₘᵢ) is the coordinates of feature point i under the coordinate system of the 3D image m; and (xₙᵢ,yₙᵢ,zₙᵢ) is the coordinates of said feature point i under the coordinate system of the 3D image n; and a unit for judging whether said mosaicing error ε is less than a threshold.

Compared with the prior art, the ultrasound 3D scanning guidance and reconstruction method and device and its system of the present invention have the following beneficial effects:

firstly, because the present invention employs multi-point scanning, it can simultaneously scan the organ at a plurality of positions and can cover the entire area of the organ, thereby obtaining a 3D image of the whole organ;

secondly, as a guidance system, the present invention can help the clinic doctors perform the scanning position by position to completely scan the whole tissue organ; and

thirdly, because the present invention can display a 3D image of the whole organ, therefore an image of any slice at any angle can be selected to diagnose a disease, thereby achieving a better effect for searching and screening some small pathological changes in an organ.

### BRIEF DESCRIPTION OF THE DRAWINGS

In order to gain a more thorough understanding of the disclosure of the present invention, reference is made to the following description in combination with the accompanying drawings, in which:

FIG. 1 is a flow chart of the ultrasound 3D scanning guidance and reconstructing method of the present invention;

FIG. 2 is a further subdivided flow chart of the reconstructing step in FIG. 1;

FIG. 3 is a further subdivided flow chart of the mosaicing step in FIG. 2;

FIG. 4 is a flow chart of the verifying step; and

FIG. 5 is a schematic illustration of the ultrasound 3D scanning guidance and reconstruction device of the present invention.

### DETAILED DESCRIPTION OF THE INVENTION

The specific embodiments of the present invention will be described in the following, but the present invention is not limited to the following specific embodiments.

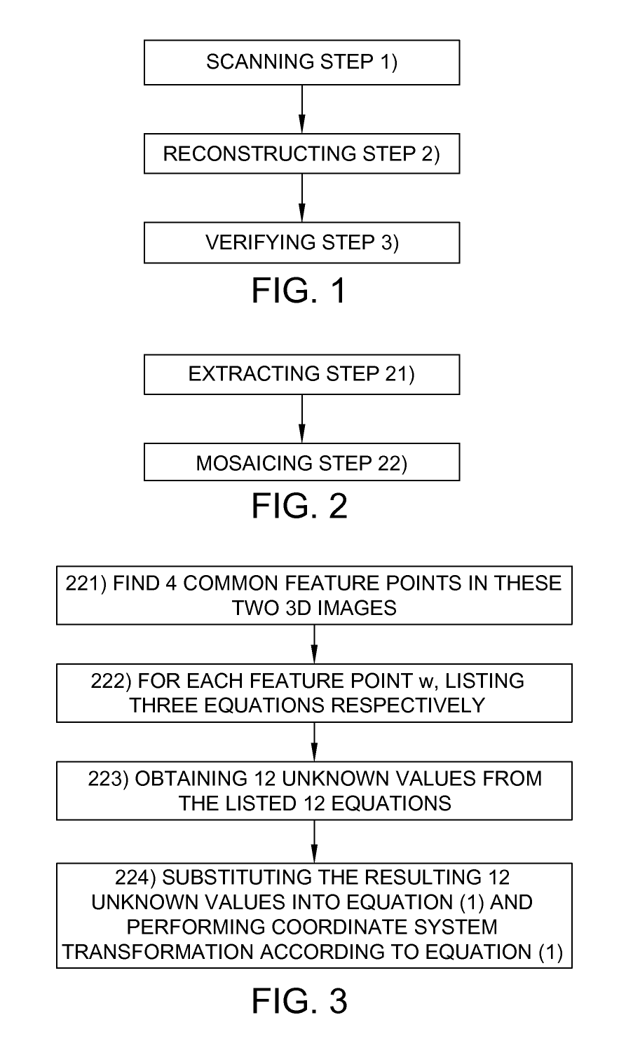

As shown in FIG. 1, an ultrasound 3D scanning guidance and reconstruction method is disclosed, which comprises: a scanning step 1): performing a multi-point scanning on an organ to obtain a plurality of 3D images, each 3D image containing corresponding feature information; a reconstructing step 2): reconstructing a 3D image of the whole organ from these 3D images.

Wherein said feature information comprises feature blood vessel, tissue modality and etc.

It can be seen from the above that the ultrasound 3D scanning guidance and reconstruction method of the present invention uses multi-point scanning, that is to simultaneously scan a plurality of points and obtain a plurality of 3D images, with each 3D image containing feature information within a range of corresponding points. Then a 3D image of the whole organ is reconstructed from these 3D images, and the resulting 3D image of the whole organ contains all the feature information of the organ. In this way, doctors can see the 3D image of the whole organ and would not miss any part of the organ.

As shown in FIG. 2, said reconstructing step 2) further comprises: an extracting step 21), for extracting said feature information of two adjacent 3D images; a mosaicing step 22), for transforming the coordinates of a point in one 3D image into coordinates under the coordinate system of the other 3D image.

Reconstructing the 3D image of the whole organ from a plurality of 3D images obtained by scanning described above employs the way of mosaicing images pairwise, i.e. mosaic two adjacent 3D images and keep mosaicing in turn until a 3D image of the whole organ is obtained. For example, consider an embodiment in which a 3-point scanning is performed, i.e. three 3D images (image 1, image 2 and image 3) are obtained. Image 1 and image 2 are first mosaiced into an image 4, and then the image 4 and image 3 are mosaiced into an image 5, which is the final resulting 3D image of the whole organ. Of course the mosaicing can be performed according to other sequences.

In order to mosaic the images, feature information in two 3D images to be mosaiced can be first extracted, such as some feature blood vessel and tissue modality of the organ and the like. And then a transformation between the coordinate systems of these two 3D images is performed, i.e. the coordinate system of one 3D image is transformed into the coordinate system of the other 3D image. If all 3D images are in a single coordinate system, the mosaicing process is completed.

As shown in FIG. 3, the mosaicing step 22) could be achieved by the following steps: 221) finding four common feature points in these two 3D images, said feature points being points in space containing feature information; 222) for each feature point W, listing the following three equations respectively:

x′_{w}=R₁₁*x_{w}+R₁₂*y_{w}+R₁₃*z_{w}+tₓ

y′_{w}=R₂₁*x_{w}+R₂₂*y_{w}+R₂₃*z_{w}+t_{y}

z′_{w}=R₃₁*x_{w}+R₃₂*y_{w}+R₃₃*z_{w}+t_{z}

wherein (x_{w},y_{w},z_{w}) is the coordinates of feature point w under the coordinate system of one 3D image, and (x′_{w},y′_{w},z′_{w}) is the coordinates of the feature point w under the coordinate system of the other 3D image;

The mosaicing step 22) also includes 223) obtaining the values of 12 unknown parameters R₁₁, R₁₂, R₁₃, R₂₁, R₂₂, R₂₃, R₃₁, R₃₂, R₃₃, tₓ, t_{y} and t_{z} from the listed 12 equations; and 224) substituting the resulting 12 unknown parameters into the following equation and performing coordinate system transformation according to the following equation:

$$[ \begin{matrix}{x}^{′} \\ {y}^{′} \\ {z}^{′} \\ 1\end{matrix} ] = [ \begin{matrix}{R}_{11} & {R}_{12} & {R}_{13} & {t}_{x} \\ {R}_{21} & {R}_{22} & {R}_{23} & {t}_{y} \\ {R}_{31} & {R}_{32} & {R}_{33} & {t}_{z} \\ 0 & 0 & 0 & 1\end{matrix} ] [ \begin{matrix}x \\ y \\ z \\ 1\end{matrix} ] \qquad ( 1 )$$

wherein (x,y,z) is the coordinates of a point on one 3D image under the coordinate system of this 3D image; and (x′,y′,z′) is the coordinates of said point under the coordinate system of the other 3D image.

It can be seen that the present invention finds the common feature points in two 3D images and calculates some coefficients required for performing the transformation between two coordinate systems by means of the common feature points and then performs the coordinate system transformation using these coefficients.

It should be pointed out that the image reconstruction method mentioned in the present invention is only one of many reconstruction algorithms, and any other image reconstruction method could occur to the skilled in the art. There are plenty of image reconstruction methods, and we only name a few herein.

As further shown in FIG. 1, the ultrasound 3D scanning guidance and reconstruction method of the present invention further comprises a verifying step 3) for verifying whether the mosaicing error of the 3D image of the whole organ resulting from mosaicing is within an acceptable range.

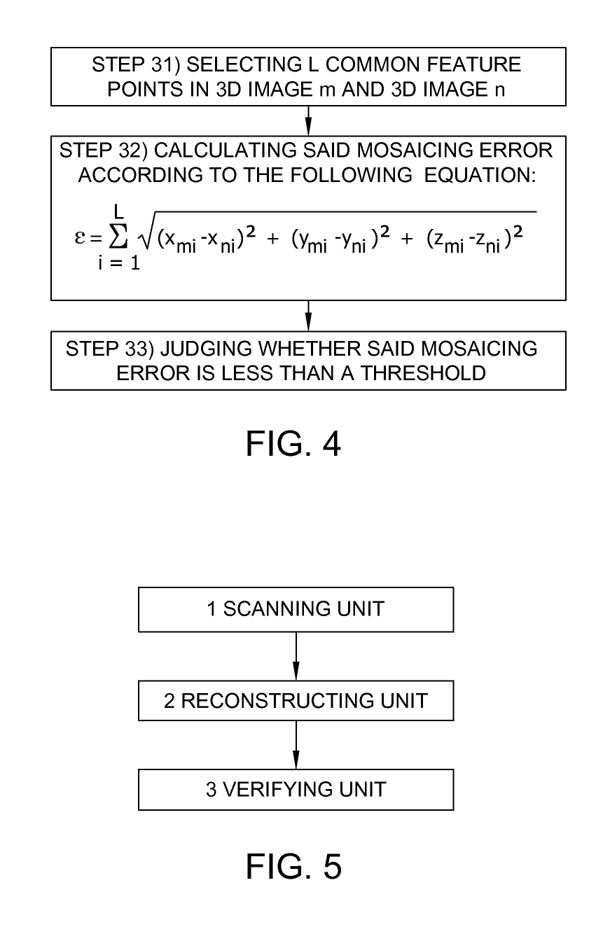

Verification of the mosaicing error could be performed by the steps shown in FIG. 4. In FIG. 4: step 31) selects L common feature points in a 3D image m and a 3D image n; step 32) calculates said mosaicing error according to the following equation:

$$ɛ = {\sum }_{i = 1}^{L} \sqrt{{( {x}_{mi} - {x}_{ni} )}^{2} + {( {y}_{mi} - {y}_{ni} )}^{2} + {( {z}_{mi} - {z}_{ni} )}^{2}}$$

wherein (xₘᵢ,yₘᵢ,zₘᵢ) is the coordinates of said feature point under the coordinate system of the 3D image m, (xₙᵢ,yₙᵢ,zₙᵢ) is the coordinates of said feature point under the coordinate system of the 3D image n and L means a total of L common feature points are selected; and step 33) judges whether said mosaicing error is less than a threshold.

In the above steps, the mosaicing error ε is calculated from the coordinates of the feature point respectively under the coordinate system of the 3D image m and under the coordinate system of the 3D image n. Of course there are other ways of verifying whether the result of mosaicing is acceptable.

As is well known, the liver is the biggest organ in human body located in the upper right corner of the abdomen and just under the right lower rib, which protects the majority of the liver. As seen from the direction facing people, average liver size of an adult is about 25 cm (left-to-right)*15 cm (front-to-back)*6 cm (up-to-down). Therefore, just from one position, it is difficult to scan the whole liver area.

In the following the technical solution of the ultrasound 3D scanning guidance and reconstruction method of the present invention is described with liver as an example.

For the liver, three points could be selected for scanning, i.e. the probes are placed in three positions: position 1, position 2 and position 3. Position 1 is in the middle of the abdomen and at the front of the left liver, at which position the probe can cover the left liver area 4, as shown in FIG. 5; position 2 is under the right lower rib, at which position the probe can cover the lower part area 5 of the right liver; and position 3 is at the side and between the fifth and sixth ribs, at which position the probe can cover the top part area 6 of the right liver. Three 3D images are obtained by scanning the liver at these three positions and each 3D image should contain some feature information.

The 3D image at position 1 should contain the following feature information: edge of left liver; middle and left branches of hepatic vein; and main branch points of hepatic artery, portal vein and bile duct.

The 3D image at position 2 should contain the following feature information: edge of lower part of right liver; main branch points of hepatic artery, portal vein and bile duct; and right branches of hepatic artery, portal vein and bile duct.

The 3D image at position 3 should contain the following feature information: edge of top part of right liver; right branches of hepatic artery, portal vein and bile duct; and middle and right branches of hepatic vein.

If the resulting 3D image from scanning does not contain corresponding feature information, then this position is rescanned to contain the corresponding feature information.

After the three 3D images containing corresponding feature information are obtained, first the 3D image at position 1 and the 3D image at position 2 are mosaiced by first finding the blood vessels and extracting their central axes. In ultrasound images the blood vessel is low echo area and therefore the low echo tubular areas are first selected and then the axes of the blood vessels are extracted pixel by pixel using area contraction.

The four common feature points in these two 3D images are: main branch points (first branch points) of hepatic artery, portal vein and bile duct (there are 3 points); and intersection points of hepatic artery, portal vein and bile duct entering the liver edge (one of three points is selected).

For each feature point (assuming four feature points are a, b, c and d), the following 12 equations are listed:

x′ₐ=R₁₁*xₐ+R₁₂*yₐ+R₁₃*zₐ+tₓ

y′ₐ=R₂₁*xₐ+R₂₂*yₐ+R₂₃*zₐ+t_{y}

z′ₐ=R₃₁*xₐ+R₃₂*yₐ+R₃₃*zₐ+t_{z}

x′_{b}=R₁₁*x_{b}+R₁₂*y_{b}+R₁₃*z_{b}+tₓ

y′_{b}=R₂₁*x_{b}+R₂₂*y_{b}+R₂₃*z_{b}+t_{y}

z′_{b}=R₃₁*x_{b}+R₃₂*y_{b}+R₃₃*z_{b}+t_{z}

x′_{c}=R₁₁*x_{c}+R₁₂*y_{c}+R₁₃*z_{c}+tₓ

y′_{c}=R₂₁*x_{c}+R₂₂*y_{c}+R₂₃*z_{c}+t_{y}

z′_{c}=R₃₁*x_{c}+R₃₂*y_{c}+R₃₃*z_{c}+t_{z}

x′_{d}=R₁₁*x_{d}+R₁₂*y_{d}+R₁₃*z_{d}+tₓ

y′_{d}=R₂₁*x_{d}+R₂₂*y_{d}+R₂₃*z_{d}+t_{y}

z′_{d}=R₃₁*x_{d}+R₃₂*y_{d}+R₃₃*z_{d}+t_{z}

The values of R11, R12, R13, R21, R22, R23, R31, R32, R33, tx, ty and tz obtained therefrom are as follows:

$$[ \begin{matrix}{R}_{11} & {R}_{12} & {R}_{13} & {t}_{x} \\ {R}_{21} & {R}_{22} & {R}_{23} & {t}_{y} \\ {R}_{31} & {R}_{32} & {R}_{33} & {t}_{z} \\ 0 & 0 & 0 & 1\end{matrix} ] = [ \begin{matrix}- 0.59 & 0.12 & 0.12 & - 80.7 \\ 0.12 & 1.11 & 0.24 & - 91.3 \\ - 0.48 & 0.21 & 0.09 & 138.6 \\ 0 & 0 & 0 & 1\end{matrix} ]$$

Please be noted that Z axis is the central axis of the probe, the positive being away from the probe and the probe surface is the origin. X axis is parallel to the probe surface and within the plane scanned by the probe, the positive being from left to light. And Y axis is perpendicular to the scanning plane, with the positive being from down to up.

Then substitute the above values into the following equation and perform a coordinate system transformation on the 3D images:

$$[ \begin{matrix}{x}^{′} \\ {y}^{′} \\ {z}^{′} \\ 1\end{matrix} ] = [ \begin{matrix}{R}_{11} & {R}_{12} & {R}_{13} & {t}_{x} \\ {R}_{21} & {R}_{22} & {R}_{23} & {t}_{y} \\ {R}_{31} & {R}_{32} & {R}_{33} & {t}_{z} \\ 0 & 0 & 0 & 1\end{matrix} ] [ \begin{matrix}x \\ y \\ z \\ 1\end{matrix} ] \qquad ( 1 )$$

Note: supposing that in the coordinate axis one pixel corresponds to one coordinate axis unit 1, and at the same time the distance of one pixel is defined as 1 mm herein, rounded after being calculated.

The 3D image obtained at position 1 and the 3D image obtained at position 2 are mosaiced to result in a 3D image′.

Next, the resulting 3D image′ is mosaiced with the 3D image obtained at position 3 and their common feature points are: a first branch point on the right branch after the hepatic artery, portal vein and bile duct enter the liver (a total of 3 points can be selected herein) and the intersection point of liver edge and hepatic vein.

For these four feature points, the above listed 12 equations are solved and we can get:

$$[ \begin{matrix}{R}_{11} & {R}_{12} & {R}_{13} & {t}_{x} \\ {R}_{21} & {R}_{22} & {R}_{23} & {t}_{y} \\ {R}_{31} & {R}_{32} & {R}_{33} & {t}_{z} \\ 0 & 0 & 0 & 1\end{matrix} ] = [ \begin{matrix}- 0.36 & 0.67 & 0.22 & 98.72 \\ 0.88 & 1.43 & 0.36 & 0.79 \\ - 0.51 & 0.02 & 0.52 & 163.28 \\ 0 & 0 & 0 & 1\end{matrix} ]$$

Then the above values are substituted into the following equation and the coordinate system transformation is performed on the 3D images:

$$[ \begin{matrix}{x}^{′} \\ {y}^{′} \\ {z}^{′} \\ 1\end{matrix} ] = [ \begin{matrix}{R}_{11} & {R}_{12} & {R}_{13} & {t}_{x} \\ {R}_{21} & {R}_{22} & {R}_{23} & {t}_{y} \\ {R}_{31} & {R}_{32} & {R}_{33} & {t}_{z} \\ 0 & 0 & 0 & 1\end{matrix} ] [ \begin{matrix}x \\ y \\ z \\ 1\end{matrix} ] \qquad ( 1 )$$

The 3D image of the whole liver is finally obtained according to the above steps.

In the following it is verified whether the mosaicing error of the resulting 3D image is acceptable or not.

For this example, the 3D image obtained at position 1 and the final resulting 3D image of the whole liver, respectively referred to as image m and image n, are selected.

Then three common points are selected from image m and image n: the first branch points on the left, middle and right branches from the direction that the hepatic vein enters the liver.

The mosaicing error are calculated according to the following equation:

$$ɛ = {\sum }_{i = 1}^{3} \sqrt{{( {x}_{mi} - {x}_{ni} )}^{2} + {( {y}_{mi} - {y}_{ni} )}^{2} + {( {z}_{mi} - {z}_{ni} )}^{2}}$$

wherein (xₘᵢ,yₘᵢ,zₘᵢ) is the coordinates of said feature point under the coordinate system of the 3D image m; and (xₙᵢ,yₙᵢ,zₙᵢ) is the coordinates of said feature point under the coordinate system of the 3D image n.

If ε is less than the threshold, then the reconstruction quality is considered to be satisfying. In this example ε is suggested to be 16. The coordinates herein all take one pixel as one unit.

In the following the spleen is taken as an example to describe the technical solution of the ultrasound 3D scanning guidance and reconstruction system of the present invention.

The spleen is located in the left hypochondriac region, between the left side of the stomach and the midriff, deep to the left ninth to eleventh ribs, with its major axis being substantially consistent with the direction of the tenth rib. Generally, splenic artery vessel and splenic vein vessel exist accompanying each other.

The probes can be placed at two positions of front-back symmetry between the ninth rib and the tenth rib (which can be referred to as a first spleen position and a second spleen position respectively), to scan the whole spleen area.

The feature information that a first 3D image obtained at the first spleen position should contain is: front edge and upper and lower edges of the spleen; splenic artery trunk and artery vessel entering the spleen; and splenic vein trunk and vein vessel entering the spleen.

The feature information that a second 3D image obtained at the second spleen position should contain is: rear edge and upper and lower edges of the spleen; splenic artery trunk and artery vessel entering the spleen; and splenic vein trunk and vein vessel entering the spleen.

In the following the reconstructing step is performed to mosaic the first 3D image with the second 3D image.

The four common feature points found in the first 3D image and the second 3D image are: first nodes on the splenic artery and vein trunk branches (two feature points); and first nodes on the second branches from front to back of the splenic artery and vein trunk branches (two feature points).

Hereby three equations are listed for each of said feature points, which are similar to the equations for the liver and won't be described any more.

The resulting values of R₁₁, R₁₂, R₁₃, R₂₁, R₂₂, R₂₃, R₃₁, R₃₂, R₃₃, tₓ, t_{y} and t_{z} are respectively:

$$[ \begin{matrix}{R}_{11} & {R}_{12} & {R}_{13} & {t}_{x} \\ {R}_{21} & {R}_{22} & {R}_{23} & {t}_{y} \\ {R}_{31} & {R}_{32} & {R}_{33} & {t}_{z} \\ 0 & 0 & 0 & 1\end{matrix} ] = [ \begin{matrix}- 0.34 & 0.06 & 0.16 & - 72.8 \\ 0.11 & 1.06 & 0.08 & 0.92 \\ - 0.55 & 0.31 & 0.11 & 137.7 \\ 0 & 0 & 0 & 1\end{matrix} ]$$

wherein X, Y and Z axes are set. Z axis is the central axis of the probe, the positive being away from the probe and the probe surface being the origin. X axis is parallel to the probe surface and within the plane scanned by the probe, the positive being from left to light. And Y axis is perpendicular to the scanning plane, with the positive being from down to up.

Then the values are substituted into the following equation:

$$[ \begin{matrix}{x}^{′} \\ {y}^{′} \\ {z}^{′} \\ 1\end{matrix} ] = [ \begin{matrix}{R}_{11} & {R}_{12} & {R}_{13} & {t}_{x} \\ {R}_{21} & {R}_{22} & {R}_{23} & {t}_{y} \\ {R}_{31} & {R}_{32} & {R}_{33} & {t}_{z} \\ 0 & 0 & 0 & 1\end{matrix} ] [ \begin{matrix}x \\ y \\ z \\ 1\end{matrix} ] \qquad ( 1 )$$

From this the 3D image of the whole spleen could be obtained.

Next the verifying process is performed, which is similar to the verifying step of the liver and won't be described any more herein.

Correspondingly, the present invention also discloses an ultrasound 3D scanning guidance and reconstruction device, as shown in FIG. 5, said ultrasound 3D scanning guidance and reconstruction device comprising: a scanning unit 1, for performing a multi-point scanning on an organ to obtain a plurality of 3D images, each 3D image containing corresponding feature information; a reconstructing unit 2, for reconstructing a 3D image of the whole organ from these 3D images.

Said feature information comprises feature blood vessel and tissue modality.

Further, said reconstructing unit 2 further comprises: an extracting unit, for extracting said feature information of two adjacent 3D images; a coordinate system transformation unit, for using said feature information, transforming the coordinates of a point in one 3D image into coordinates under the coordinate system of the other 3D image.

Wherein said coordinate system transformation unit further comprises: a unit for finding four common feature points in these two 3D images, said feature points being points in space containing feature information; a unit for listing the following three equations respectively for each feature point W:

x′_{w}=R₁₁*x_{w}+R₁₂*y_{w}+R₁₃*z_{w}+tₓ

y′_{w}=R₂₁*x_{w}+R₂₂*y_{w}+R₂₃*z_{w}+t_{y}

z′_{w}=R₃₁*x_{w}+R₃₂*y_{w}+R₃₃*z_{w}+t_{z}

wherein (x_{w},y_{w},z_{w}) is the coordinates of feature point w under the coordinate system of one 3D image, and (x′_{w},y′_{w},z′_{w}) is the coordinates of the feature point w under the coordinate system of the other 3D image;

The coordinate system transformation unit also a unit for obtaining the values of 12 unknown parameters R₁₁, R₁₂, R₁₃, R₂₁, R₂₂, R₂₃, R₃₁, R₃₂, R₃₃, tₓ, t_{y} and t_{z} from the listed 12 equations; and a unit for substituting the resulting 12 unknown parameters into the following equation and performing coordinate system transformation according to the following equation:

$$[ \begin{matrix}{x}^{′} \\ {y}^{′} \\ {z}^{′} \\ 1\end{matrix} ] = [ \begin{matrix}{R}_{11} & {R}_{12} & {R}_{13} & {t}_{x} \\ {R}_{21} & {R}_{22} & {R}_{23} & {t}_{y} \\ {R}_{31} & {R}_{32} & {R}_{33} & {t}_{z} \\ 0 & 0 & 0 & 1\end{matrix} ] [ \begin{matrix}x \\ y \\ z \\ 1\end{matrix} ] \qquad ( 1 )$$

wherein (x,y,z) is the coordinates of a point on one 3D image under the coordinate system of this 3D image; and (x′,y′,z′) is the coordinates of said point under the coordinate system of the other 3D image.

As further shown in FIG. 5, the ultrasound 3D scanning guidance and reconstruction device of the present invention further comprises a verifying unit 3 for verifying whether the mosaicing error of the 3D image of the whole organ resulting from mosaicing is within an acceptable range.

Said verifying unit 3 further comprises: a unit for selecting L common feature points in a 3D image m and a 3D image n; a unit for calculating said mosaicing error according to the following equation:

$$ɛ = {\sum }_{i = 1}^{L} \sqrt{{( {x}_{mi} - {x}_{ni} )}^{2} + {( {y}_{mi} - {y}_{ni} )}^{2} + {( {z}_{mi} - {z}_{ni} )}^{2}}$$

wherein (xₘᵢ,yₘᵢ,zₘᵢ) is the coordinates of feature point i under the coordinate system of the 3D image m; and (xₙᵢ,yₙᵢ,zₙᵢ) is the coordinates of said feature point i under the coordinate system of the 3D image n; and a unit for judging whether said mosaicing error ε is less than a threshold.

Since the technical solution of the ultrasound 3D scanning guidance and reconstruction device of the present invention corresponds to the technical solution of the ultrasound 3D scanning guidance and reconstruction method of the present invention, the technical solution of the ultrasound 3D scanning guidance and reconstruction device of the present invention won't be described in detail herein.

Moreover, the present invention also discloses an ultrasound system, which comprises an ultrasound 3D scanning guidance and reconstruction device, said ultrasound 3D scanning guidance and reconstruction device comprising: a scanning unit 1, for performing a multi-point scanning on an organ to obtain a plurality of 3D images, each 3D image containing corresponding feature information; a reconstructing unit 2, for reconstructing a 3D image of the whole organ from these 3D images.

Said feature information comprises feature blood vessel and tissue modality.

Said reconstructing unit 2 further comprises: an extracting unit, for extracting said feature information of two adjacent 3D images; a coordinate system transformation unit, for using said feature information, transforming the coordinates of a point in one 3D image into coordinates under the coordinate system of the other 3D image.

Wherein said coordinate system transformation unit further comprises: a unit for finding four common feature points in these two 3D images, said feature points being points in space containing feature information; a unit for listing the following three equations respectively for each feature point W:

x′_{w}=R₁₁*x_{w}+R₁₂*y_{w}+R₁₃*z_{w}+tₓ

y′_{w}=R₂₁*x_{w}+R₂₂*y_{w}+R₂₃*z_{w}+t_{y}

z′_{w}=R₃₁*x_{w}+R₃₂*y_{w}+R₃₃*z_{w}+t_{z}

wherein (x_{w},y_{w},z_{w}) is the coordinates of feature point w under the coordinate system of one 3D image, and (x′_{w},y′_{w},z′_{w}) is the coordinates of the feature point w under the coordinate system of the other 3D image;

The coordinate system transformation unit also includes a unit for obtaining the values of 12 unknown parameters R₁₁, R₁₂, R₁₃, R₂₁, R₂₂, R₂₃, R₃₁, R₃₂, R₃₃, tₓ, t_{y} and t_{z} from the listed 12 equations; and a unit for substituting the resulting 12 unknown parameters into the following equation and performing coordinate system transformation according to the following equation:

$$[ \begin{matrix}{x}^{′} \\ {y}^{′} \\ {z}^{′} \\ 1\end{matrix} ] = [ \begin{matrix}{R}_{11} & {R}_{12} & {R}_{13} & {t}_{x} \\ {R}_{21} & {R}_{22} & {R}_{23} & {t}_{y} \\ {R}_{31} & {R}_{32} & {R}_{33} & {t}_{z} \\ 0 & 0 & 0 & 1\end{matrix} ] [ \begin{matrix}x \\ y \\ z \\ 1\end{matrix} ] \qquad ( 1 )$$

wherein (x,y,z) is the coordinates of a point on one 3D image under the coordinate system of this 3D image; and (x′,y′,z′) is the coordinates of said point under the coordinate system of the other 3D image.

The ultrasound system further comprises a verifying unit 3 for verifying whether the mosaicing error of the 3D image of the whole organ resulting from mosaicing is within an acceptable range.

Said verifying unit 3 further comprises: a unit for selecting L common feature points in a 3D image m and a 3D image n; a unit for calculating said mosaicing error according to the following equation:

$$ɛ = {\sum }_{i = 1}^{L} \sqrt{{( {x}_{mi} - {x}_{ni} )}^{2} + {( {y}_{mi} - {y}_{ni} )}^{2} + {( {z}_{mi} - {z}_{ni} )}^{2}}$$

wherein (xₘᵢ,yₘᵢ,zₘᵢ) is the coordinates of feature point i under the coordinate system of the 3D image m; and (xₙᵢ,yₙᵢ,zₙᵢ) is the coordinates of said feature point i under the coordinate system of the 3D image n; and a unit for judging whether said mosaicing error ε is less than a threshold.

Of course, the technical solution of the ultrasound 3D scanning guidance and reconstructing device and method can be applied in any ultrasound system.

Although the specific embodiments of the present invention have been described in combination with the accompanying drawings, the skilled in the art could make various changes, modifications and equivalent substitutions to the present invention without departing from the spirit and the scope of the present invention. These changes, modifications and equivalent substitutions are all intended to fall within the spirit and scope defined by the following claims.

1. An ultrasound 3D scanning guidance and reconstruction method, comprising:
performing a multi-point scan of an organ to obtain a plurality of first 3D images, each first 3D image containing corresponding feature information; and
reconstructing a second 3D image of the whole organ from the plurality of first 3D images.

2. The ultrasound 3D scanning guidance and reconstruction method of claim 1, wherein the feature information is one or more of the following: a feature blood vessel and a tissue modality.

3. The ultrasound 3D scanning guidance and reconstruction method of claim 2, wherein reconstructing further comprises:
extracting the feature information of two adjacent first 3D images of the plurality of first 3D images;
transforming the coordinates of a point in a first 3D image of the two adjacent first 3D images into coordinates under the coordinate system of a second 3D image of the two adjacent first 3D images, the transformation using the feature information; and
repeating the above steps until all of the plurality of first 3D images are under a single coordinate system.

4. The ultrasound 3D scanning guidance and reconstruction method of claim 3, wherein transforming comprises:
finding four common feature points in the two adjacent first 3D images, the feature points being points in space containing feature information;
for each feature point W, listing the following three equations respectively:
x′_{w}=R₁₁*x_{w}+R₁₂*y_{w}+R₁₃*z_{w}+tₓ
y′_{w}=R₂₁*x_{w}+R₂₂*y_{w}+R₂₃*z_{w}+t_{y}
z′_{w}=R₃₁*x_{w}+R₃₂*y_{w}+R₃₃*z_{w}+t_{z}
wherein (x_{w},y_{w},z_{w}) is the coordinates of feature point w under the coordinate system of the first 3D image of the two adjacent first 3D images, and (x′_{w},y′_{w},z′_{w}) is the coordinates of said feature point w under the coordinate system of the second 3D image of the two adjacent first 3D images;
obtaining the values of a plurality of unknown parameters R₁₁, R₁₂, R₁₃, R₂₁, R₂₂, R₂₃, R₃₁, R₃₂, R₃₃, tₓ, t_{y} and t_{z} from the listed equations;
substituting the resulting plurality of unknown parameters into the following equation and performing coordinate system transformation according to the following equation:
$$[ \begin{matrix}{x}^{′} \\ {y}^{′} \\ {z}^{′} \\ 1\end{matrix} ] = [ \begin{matrix}{R}_{11} & {R}_{12} & {R}_{13} & {t}_{x} \\ {R}_{21} & {R}_{22} & {R}_{23} & {t}_{y} \\ {R}_{31} & {R}_{32} & {R}_{33} & {t}_{z} \\ 0 & 0 & 0 & 1\end{matrix} ] [ \begin{matrix}x \\ y \\ z \\ 1\end{matrix} ]$$
wherein (x,y,z) is the coordinates of a point on the first 3D image of the two adjacent first 3D images under the coordinate system of the first 3D image; and (x′,y′,z′) is the coordinates of said point under the coordinate system of the second 3D image of the two adjacent first 3D images.

5. The ultrasound 3D scanning guidance and reconstruction method of claim 4, wherein further comprising verifying whether the mosaicing error of the second 3D image of the whole organ resulting from mosaicing is within an acceptable range.

6. The ultrasound 3D scanning guidance and reconstruction method of claim 5, wherein verifying further comprises:
selecting L common feature points in a 3D image m and a 3D image n;
calculating the mosaicing error according to the following equation:
$$ɛ = {\sum }_{i = 1}^{L} \sqrt{{( {x}_{mi} - {x}_{ni} )}^{2} + {( {y}_{mi} - {y}_{ni} )}^{2} + {( {z}_{mi} - {z}_{ni} )}^{2}}$$
wherein (xₘᵢ,yₘᵢ,zₘᵢ) is the coordinates of feature point i under the coordinate system of the 3D image m; and (xₙᵢ,yₙᵢ,zₙᵢ) is the coordinates of said feature point i under the coordinate system of the 3D image n; and
judging whether the mosaicing error ε is less than a threshold.

7. An ultrasound 3D scanning guidance and reconstruction device, comprising:
a scanning unit, configured to perform a multi-point scan of an organ to obtain a plurality of first 3D images, each first 3D image containing corresponding feature information; and
a reconstructing unit, configured to reconstruct a second 3D image of the whole organ from the plurality of first 3D images.

8. The ultrasound 3D scanning guidance and reconstruction device of claim 7, wherein the feature information is one or more of the following: a feature blood vessel and a tissue modality.

9. The ultrasound 3D scanning guidance and reconstruction device of claim 8, wherein said reconstructing unit further comprises:
an extracting unit, configured to extract the feature information of two adjacent 3D images of the plurality of first 3D images;
a coordinate system transformation unit, configured to use the feature information, to transform the coordinates of a point in a first 3D image of the two adjacent first 3D images into coordinates under the coordinate system of a second 3D image of the two adjacent first 3D images.

10. The ultrasound 3D scanning guidance and reconstruction device of claim 9, wherein said coordinate system transformation unit further comprises:
a unit configured to determine four common feature points in the two adjacent first 3D images, the feature points being points in space containing feature information;
a unit configured to list the following three equations respectively for each feature point W:
x′_{w}=R₁₁*x_{w}+R₁₂*y_{w}+R₁₃*z_{w}+tₓ
y′_{w}=R₂₁*x_{w}+R₂₂*y_{w}+R₂₃*z_{w}+t_{y}
z′_{w}=R₃₁*x_{w}+R₃₂*y_{w}+R₃₃*z_{w}+t_{z}
wherein (x_{w},y_{w},z_{w}) is the coordinates of feature point w under the coordinate system of one 3D image, and (x′_{w},y′_{w},z′_{w}) is the coordinates of the feature point w under the coordinate system of the other 3D image;
a unit configured to calculate the values of a plurality of unknown parameters R₁₁, R₁₂, R₁₃, R₂₁, R₂₂, R₂₃, R₃₁, R₃₂, R₃₃, tₓ, t_{y} and t_{z} from the listed equations; and
a unit configured to substitute the resulting plurality of unknown parameters into the following equation and performing coordinate system transformation according to the following equation:
$$[ \begin{matrix}{x}^{′} \\ {y}^{′} \\ {z}^{′} \\ 1\end{matrix} ] = [ \begin{matrix}{R}_{11} & {R}_{12} & {R}_{13} & {t}_{x} \\ {R}_{21} & {R}_{22} & {R}_{23} & {t}_{y} \\ {R}_{31} & {R}_{32} & {R}_{33} & {t}_{z} \\ 0 & 0 & 0 & 1\end{matrix} ] [ \begin{matrix}x \\ y \\ z \\ 1\end{matrix} ]$$
wherein (x,y,z) is the coordinates of a point on the first 3D image of the two adjacent first 3D images under the coordinate system of the first 3D image; and (x′,y′,z′) is the coordinates of said point under the coordinate system of the second 3D image of the two adjacent first 3D images.

11. The ultrasound 3D scanning guidance and reconstruction device of claim 10, further comprising: a verifying unit, configured to verify whether a mosaicing error of the second 3D image of the whole organ resulting from mosaicing is within an acceptable range.

12. The ultrasound 3D scanning guidance and reconstruction device of claim 11, wherein said verifying unit further comprises:
a unit configured to select L common feature points in a 3D image m and a 3D image n;
a unit configured to calculate the mosaicing error according to the following equation:
$$ɛ = {\sum }_{i = 1}^{L} \sqrt{{( {x}_{mi} - {x}_{ni} )}^{2} + {( {y}_{mi} - {y}_{ni} )}^{2} + {( {z}_{mi} - {z}_{ni} )}^{2}}$$
wherein (xₘᵢ,yₘᵢ,zₘᵢ) is the coordinates of feature point i under the coordinate system of the 3D image m; and (xₙᵢ,yₙᵢ,zₙᵢ) is the coordinates of said feature point i under the coordinate system of the 3D image n; and
a unit configured to determine whether the mosaicing error ε is less than a threshold.

13. An ultrasound system, comprising;
an ultrasound 3D scanning guidance and reconstruction device, comprising:
a scanning unit, configured to perform a multi-point scan of an organ to obtain a plurality of first 3D images, each first 3D image containing corresponding feature information; and
a reconstructing unit, configured to reconstruct a second 3D image of the whole organ from the plurality of first 3D images.

14. The ultrasound system of claim 13, wherein the feature information is one or more of the following: a feature blood vessel and a tissue modality.

15. The ultrasound system of claim 14, wherein said reconstructing unit further comprises:
an extracting unit, configured to extract the feature information of two adjacent 3D images of the plurality of first 3D images;
a coordinate system transformation unit, configured to use the feature information, to transform the coordinates of a point in a first 3D image of the two adjacent first 3D images into coordinates under the coordinate system of a second 3D image of the two adjacent first 3D images.

16. The ultrasound system of claim 15, wherein said coordinate system transformation unit further comprises:
a unit configured to determine four common feature points in the two adjacent first 3D images, the feature points being points in space containing feature information;
a unit configured to list the following three equations respectively for each feature point W:
x′_{w}=R₁₁*x_{w}+R₁₂*y_{w}+R₁₃*z_{w}+tₓ
y′_{w}=R₂₁*x_{w}+R₂₂*y_{w}+R₂₃*z_{w}+t_{y}
z′_{w}=R₃₁*x_{w}+R₃₂*y_{w}+R₃₃*z_{w}+t_{z}
wherein (x_{w},y_{w},z_{w}) is the coordinates of feature point w under the coordinate system of one 3D image, and (x′_{w},y′_{w},z′_{w}) is the coordinates of the feature point w under the coordinate system of the other 3D image;
a unit configured to calculate the values of a plurality of unknown parameters R₁₁, R₁₂, R₁₃, R₂₁, R₂₂, R₂₃, R₃₁, R₃₂, R₃₃, tₓ, t_{y} and t_{z} from the listed equations; and
a unit configured to substitute the resulting plurality of unknown parameters into the following equation and performing coordinate system transformation according to the following equation:
$$[ \begin{matrix}{x}^{′} \\ {y}^{′} \\ {z}^{′} \\ 1\end{matrix} ] = [ \begin{matrix}{R}_{11} & {R}_{12} & {R}_{13} & {t}_{x} \\ {R}_{21} & {R}_{22} & {R}_{23} & {t}_{y} \\ {R}_{31} & {R}_{32} & {R}_{33} & {t}_{z} \\ 0 & 0 & 0 & 1\end{matrix} ] [ \begin{matrix}x \\ y \\ z \\ 1\end{matrix} ]$$
wherein (x,y,z) is the coordinates of a point on the first 3D image of the two adjacent first 3D images under the coordinate system of the first 3D image; and (x′,y′,z′) is the coordinates of said point under the coordinate system of the second 3D image of the two adjacent first 3D images.

17. The ultrasound system of claim 16, further comprising: a verifying unit, configured to verify whether a mosaicing error of the second 3D image of the whole organ resulting from mosaicing is within an acceptable range.

18. The ultrasound system of claim 17, wherein said verifying unit further comprises:
a unit configured to select L common feature points in a 3D image m and a 3D image n;
a unit configured to calculate the mosaicing error according to the following equation:
$$ɛ = {\sum }_{i = 1}^{L} \sqrt{{( {x}_{mi} - {x}_{ni} )}^{2} + {( {y}_{mi} - {y}_{ni} )}^{2} + {( {z}_{mi} - {z}_{ni} )}^{2}}$$
wherein (xₘᵢ,yₘᵢ,zₘᵢ) is the coordinates of feature point i under the coordinate system of the 3D image m; and (xₙᵢ,yₙᵢ,zₙᵢ) is the coordinates of said feature point i under the coordinate system of the 3D image n; and
a unit configured to determine whether the mosaicing error ε is less than a threshold.

19. The ultrasound 3D scanning guidance and reconstruction method of claim 1, wherein performing a multi-point scan comprises simultaneously scanning the organ at a plurality of positions.

20. The ultrasound 3D scanning guidance and reconstruction device of claim 7, wherein said scanning unit is configured to simultaneously scan the organ at a plurality of positions.
